(19) United States
(12) Patent Application Publication
(10) Pub. No.: US 2003/0129243 A1
(43) Pub. Date: Jul. 10, 2003

(54) Compositions comprising nucleic acids incorporated in bilaminar mineral particles

(76) Inventor: Bruno Pitard, Reze (FR)

Correspondence Address: ROSS J. OEHLER, AVENTIS PHARMACEUTICALS INC., ROUTE 202-206, MAIL CODE: D303A, BRIDGEWATER, NJ, 08807, US

(21) Appl. No.: 10/169,392

(22) PCT Filed: Dec. 27, 2000
(86) PCT No.: PCT/FR00/03702

(30) Foreign Application Priority Data
Dec. 30, 1999 (FR) 99/16707

(51) Int. Cl. A61K 48/00; C12N 15/87; A61K 9/14

(52) U.S. Cl. 424/489; 514/44; 435/459

(57) ABSTRACT

The invention concerns compositions comprising at least a nucleic acid and a mineral particle having an interchangeable foliate structure, the method for preparing them and their uses for in vivo, in vitro and/or ex vivo nucleic acid transfection. Said mineral particles preferably correspond to general formula (I):

[M^{II}₁₋ₓM^{III}ₓ(OH)₂]^{x+}[X^{m-}_{x/m}nH₂O]^{x-}.

The present invention relates to compositions comprising at least one nucleic acid and one mineral particle having an exchangeable lamellae structure, their method of preparation and their applications for the transfection in vivo, in vitro and/or ex vivo of nucleic acids.

With the development of biotechnology, the possibility of efficiently transferring nucleic acids into cells (i.e. of introducing them into cells in such a way that the gene of interest that they are carrying is expressed therein) has become a basic technique with many biotechnological applications. It may involve the transfer of nucleic acids into cells in vitro, for example for the production of recombinant proteins, or in the laboratory for studying the regulation of gene expression, the cloning of genes or any other manipulation involving DNA. It may also involve the transfer of nucleic acids into cells in vitro, for example for the production of vaccines, for labelling studies or also for therapeutic approaches It may alternatively involve the transfer of genes ex vivo into cells which have been removed from an organism with a view to their subsequent readministration, for example for the creation of transgenic animals.

To date, many methods for transferring nucleic acids into cells exist: calcium phosphate precipitation, electroporation, microinjection, viral infection, injection of naked DNA, or alternatively the use of non-viral vectors such as for example cationic polymers, biochemical vectors (consisting of a cationic protein associated with a cellular receptor), or alternatively lipofectants, and more particularly cationic lipids.

However, these techniques developed to date have many disadvantages which limit the efficiency of the transfection and do not make it possible to satisfactorily resolve the difficulties which are linked to the transfer of genes into cells and/or the organism. Thus, calcium phosphate precipitation is a method which is quite inefficient, in particular in relation to the injection of naked DNA, and it has, therefore, been abandoned for a long time. While it has been shown that the injection of “naked” nucleic acids, i.e. non-formulated, makes it possible to obtain acceptable levels of transfection in certain cell types in vivo (see in particular Patent Application WO 90/11092), the naked nucleic acids enjoy, however, only a short plasma half-life, because of their degradation by enzymes and their elimination via the urinary tracts. The level of efficiency of gene transfer by injection of a naked nucleic acid remains too low for most of the applications envisaged. The use of polymers or cationic lipids thus constitutes a possible remedy with regard to enzymatic degradations undergone by the DNA to be transfected, but it has often been observed that their use inhibits more or less strongly the efficiency of transfection in muscles and that they induce, in addition, toxicity with regard to cells. While recombinant viruses make it possible to obtain a good efficiency of transfer of nucleic acids, their use presents, however, certain risks which are linked to their viral nature such as pathogenicity, transmission, replication, recombination, transformation, immunogenicity, etc. It is, therefore, preferable to avoid the techniques of viral transfection. Finally, techniques such as electroporation constitute an alternative which is of value, but limited to a local administration. They quite often provoke irreversible damage to the cell membranes. It appears, therefore, that it would be desirable to have a transfer vector which makes it possible both to protect the nucleic acids from the enzymatic degradations and to obtain a satisfactory efficiency of transfer into the cells, without, for all that, damaging them or inducing toxicity.

The present invention provides an advantageous solution to these problems The Applicant has in point of fact shown that the compositions comprising a nucleic acid and a mineral particle having an exchangeable lamellae structure enable the transfer in vitro, ex vivo or in vivo of the said nucleic acid into a cell and/or an organ with an efficiency which is greater than those obtained to date with the conventional techniques of non-viral transfer. The compositions of,the invention make it possible, in addition, to avoid the disadvantages which are linked to the use of viral vectors or physical techniques of transfection.

Thus, a first subject of the present invention relates to the compositions comprising at least one nucleic acid and one mineral particle having an exchangeable lamellae structure.

For the purposes of the invention, by “mineral particles having an exchangeable lamellae structure” is meant mineral particles whose structure is based on a stack of lamellae of composition M(OH)₂, analogous to those which are well known of brucite (Mg(OH)₂), M representing a divalent metal, but in which a part of the divalent metals is replaced with trivalent metals in such a way as to render the lamellae cationic overall. The inter-lamellae spaces comprise anionic entities which are solvated by water molecules. Such a structure is represented in FIG. 1 and is also called “lamellar double hydroxide”. These particles have as a general formula:

[M₁₋ₓ^{II}Mₓ^{III}(OH)₂]^{x+}[X_{x/m}^{m-}.nH₂O[^{x-}

in which M^{II} represents a divalent metal cation, M^{III} represents a trivalent metal cation, X^{m-} represents an interfoliar anion and m is an integer greater than or equal to 1, x is strictly between 0 and 1, and n is strictly greater than 0.

Such mineral particles have already been described for applications in many domains such as catalysis or the environment. Specifically, they form nanostructured materials where molecular or colloidal species are intercalated in a lamellar host structure. The properties of these structures may thus be exploited in heterogeneous or homogeneous catalysis on a support, in techniques of exchange and separation of in particular optical isomers, for the design of membranes, possibly selective, for filtration and permeation, for the trapping and controlled restoration of molecules, or alternatively for the design of electroactive materials and deposits, electrodes and electronic devices.

Thus, the article Choy et al (1999, J. Am. Chem. Soc. 121(6), 1399-1400) describes the preparation of hybrid nanoparticles composed of sheets of a lamellar double hydroxide (LDH), pristine (Mg₂Al(NO₃)-LDH), between which there are intercalated nucleoside monophosphates or DNA fragments of herring testicles. The nucleic molecules intercalated between the particles described in this article have a size of less than 1 000 base pairs, No use of these molecules is described.

However, such mineral particles had never been used for the transfer of nucleic acids and there was nothing to suggest that it would be possible to obtain a very clear improvement of the efficiency of transfer, in particular with respect to the results of transfection obtained by injection of naked DNA or DNA formulated with conventional synthetic vectors such as cationic lipids.

The mineral particles having an exchangeable lamellae structure which may be used in the context of the present invention are often named according to the nature of the major metal cations of the lamellae: for example hydrotalcite (Mg—Al), pyroaurite (Mg—Fe), stichtite (Mg—Cr) or alternatively takovite (Ni—Al). The general formula indicated above underlines the great variety of the mineral particles that it is possible to prepare by varying the nature of the two metal cations M^{II} and M^{III}, their respective proportions with possible extension to more than two types of cations (for example Mg, Zn and Al), the nature of the interfoliar anions, or alternatively the hydration state. In addition, the richness of the general formula indicated above is further increased by a structural diversity which results from the existence of polytypes which differ in the stacking sequence of the lamellae and in the appearance of superstructures due for example to a cationic classification in the hydroxylated lamella.

The M^{II} divalent metal cations may be chosen for example from magnesium (Mg), chromium (Cr), manganese (Mn), iron (Fe), nickel (Ni), cobalt (Co), copper (Cu), calcium (Ca) or alternatively zinc (Zn). Preferably, the divalent metal cation is magnesium. The M^{III} trivalent metal cations may be chosen for example from aluminium (Al), chromium (Cr), manganese (Mn), iron (Fe), nickel (Ni), cobalt (Co) or alternatively gallium (Ga). Preferably, the trivalent metal cation is aluminium. In addition, the two members of the pair M^{II} and M^{III} may also correspond to the same element (for example Fe^{II}/Fe^{III} or Mn^{II}/M^{III}).

The X^{m-} interfoliar anions may be chosen for example from halides, oxoanions, iso- and heteroanions, complex anions, or alternatively organic anions. Examples which may be mentioned are fluorides, chlorides, bromides, carbonates, nitrates, sulphates or alternatively tetrahedral oxoanions such as CrO₄²⁻. Preferably, the anion is chosen from CO₃²⁻ carbonates.

The value of x is strictly between 0 and 1, i.e. 0 and 1 are excluded values. Preferably, x is between 0.05 et 0.95, and preferably between 0.1 and 0.9. Even more preferably, x is between 0.2 et 0.85. As an example, x may be equal to 0.25. As far as the value of n is concerned, this indicates the hydration state of the particle in question and it is strictly greater than 0. Preferably, n is greater than or equal to 0.1. For example, n may be equal to 0.5. m is an integer greater than or equal to 1 which represents the charge of the interfoliar anion. For example, m may be equal to 1, 2, 3, 4, 5 or 6.

Mineral particles having an exchangeable lamellae structure according to the present invention may be chosen for example from hydrotalcite of formula Mg₆Al₂CO₃(OH)₁₆.4H₂O, manasseite of formula Mg₆Al₂CO₃(OH)₁₆.4H₂O, pyroaurite of formula Mg₆Fe₂CO₃(OH)₁₆.4H₂O, stichtite of formula Mg₆Cr₂CO₃(OH)₁₆.4H₂O, takovite of formula Ni₆Al₂CO₃(OH)₁₆.4H₂O, reevesite of formula Ni₆Fe₂CO₃(OH)₁₆.4H₂O, or alternatively comblainite of formula Ni₆CO₂CO₃(OH)₁₆.4H₂O. Other mineral particles having an exchangeable lamellae structure may also be suitable.

Preferably, the compositions according to the present invention comprise at least one nucleic acid and hydrotalcite of formula Mg₆Al₂CO₃(OH)₁₆.4H₂O.

Mineral particles having an exchangeable lamellae structure according to the present invention are either commercial, or they may be prepared according to methods which are similar to the known methods termed “soft chemistry”. In particular, the preparation of the mineral particles according to the invention may be based on the controlled precipitation of solutions or aqueous suspensions containing both the metal cations which are intended to be placed in the hydroxylated framework and the anions which are intended to occupy the interlamellar domains. Under these conditions, there is construction simultaneously of the interlamellar domains consisting of anions and water molecules, and of the lamella. The nature and texture of the phases obtained are highly dependent on the procedure conditions (temperature, speed of addition and concentration of the reagents, control of pH), but also on the geometry of the reactor and on the prior state of association of the metal cations in the reagents (existence of oligomers). The method of preparation always in the end comes down to putting together, at adequate concentration and reactivity, the species which lead to the construction of the mineral particles under optimal conditions. These optimal conditions are determined case by case according to the conventional method of trial and error.

For the purposes of the invention, by “nucleic acid” is meant both a deoxyribonucleic acid and a ribonucleic acid. They may be natural or artificial sequences, and in particular genomic DNA (gDNA), complementary DNA (cDNA), messenger RNA (mRNA), transfer RNA (tRNA), ribosomal RNA (rRNA), hybrid sequences or synthetic or semisynthetic sequences, oligonucleotides which are modified or otherwise. These nucleic acids may be for example of human, animal, plant, bacterial or viral origin. They may be obtained by any technique known to persons skilled in the art, and in particular by the screening of libraries, by chemical synthesis or alternatively by mixed methods including the chemical or enzymatic modification of sequences obtained by the screening of libraries. They may be chemically modified.

Concerning more particularly the deoxyribonucleic acids, they may be single- or double-stranded, or alternatively short oligonucleotides or longer sequences. In particular, the nucleic acids advantageously consist of for example plasmids, vectors, episomes or expression cassettes. These deoxyribonucleic acids may in particular carry an origin of replication which is functional or otherwise in the target cell, one or more marker genes, sequences for regulating transcription or replication, genes of therapeutic interest, antisense sequences which are modified or otherwise or alternatively regions for binding to other cellular components.

Preferably, the said nucleic acid has a size greater than 1 000 base pairs. Still more preferably, it has a size of between about 1 000 base pairs and about 2 000 000 base pairs (2 Mb), about 1 000 base pairs and about 100 000 base pairs (100 kb), and about 1 000 base pairs and about 20 000 base pairs (20 kb).

Advantageously, the nucleic acid comprises one or more genes of therapeutic interest under the control of regulatory sequences, for example one or more promoters and a transcriptional terminator which are active in the target cells.

For the purpose of the invention, by gene of therapeutic interest is meant in particular any gene encoding a protein product which has a therapeutic effect. The protein product thus encoded may be in particular a protein or a peptide. This protein product may be exogenous homologous or endogenous with regard to the target cell, i.e. a product which is normally expressed in the target cell when this cell has no pathological condition. In this case, the expression of a protein makes it possible for example to offset an insufficient expression in the cell or the expression of a protein which is inactive or weakly active because of a modification, or alternatively to overexpress the said protein. The gene of therapeutic interest may also encode a mutant of a cellular protein which has for example an increased stability or a modified activity. The protein product may also be heterologous with regard to the target cell. In this case, an expressed protein may for example supplement or provide an activity which is deficient in the cell, allowing it to combat a pathological condition, or to stimulate an immune response.

Among the therapeutic products, for the purpose of the present invention, mention may be made more particularly of enzymes, blood derivatives, hormones, lymphokines (for example interleukins, interferons or TNF: see FR 92/03120), growth factors, neurotransmitters or precursors thereof or synthesis enzymes, trophic factors (for example BDNF, CNTF, NGF, IGF, GMF, aFGF, bFGF, VEGF, NT3, NT5 or HARP/pleiotrophin), apolipoproteins (for example ApoAI, ApoAIV or ApoE: see FR 93/05125), dystrophin or a minidystrophin (FR 91/11947), the CFTR protein which is associated with cystic fibrosis, tumour suppressor genes (for example p53, Rb, Rap1A, DCC or k-rev: see FR 93/04745), genes encoding factors which are involved in coagulation (Factors VII, VIII, IX), the genes involved in DNA repair, suicide genes (thymidine kinase, cytosine deaminase), the genes for haemoglobin, or other transporter proteins, metabolic enzymes or catabolic enzymes.

The nucleic acid of therapeutic interest may also be a gene or an antisense sequence, whose expression in the target cell makes it possible to control the expression of genes or the transcription of cellular mRNAs. Such sequences may, for example, be transcribed in the target cell into RNAs which are complementary to cellular mRNAs and thus block their translation into protein, according to the technique described in Patent EP 140 308. The therapeutic genes also comprise the sequences encoding ribozymes, which are capable of selectively destroying target RNAs (EP 321 201).

As indicated above, the nucleic acid may also comprise one or more genes encoding an antigenic peptide, which is capable of generating an immune response in humans or in animals. In this specific embodiment, the invention enables the production either of vaccines or of immunotherapeutic treatments applied to humans or to animals, in particular against microorganisms, viruses or cancers. They may be in particular antigenic peptides specific for the Epstein Barr virus, the HIV virus, the hepatitis B virus (EP 185 573), the pseudo-rabies virus, the syncitia forming virus, other viruses or alternatively antigenic peptides specific for tumours (EP 259 212).

As indicated above, the nucleic acid preferably also comprises sequences enabling the expression of the gene of therapeutic interest and/or of the gene encoding the antigenic peptide in the desired cell or organ. They may be the sequences which are responsible naturally for the expression of the gene considered when these sequences are capable of functioning in the infected cell. They may also be sequences of different origin (which are responsible for the expression of other proteins, or are even synthetic). In particular, they may be promoter sequences of eukaryotic or viral genes. For example, they may be promoter sequences derived from the genome of the cell whose infection is desired. Similarly, they may be promoter sequences derived from the genome of a virus. In this regard, mention may be made of for example the promoters of the E1A, MLP, CMV, RSV or HSV genes. In addition, these expression sequences may be modified for example by addition of activating or regulatory sequences. They may also be inducible or repressible promoters.

Moreover, the nucleic acid may also comprise, in particular upstream of the gene of therapeutic interest, a signal sequence which directs the synthesized therapeutic product in the secretory pathways of the target cell. This signal sequence may be the natural signal sequence of the therapeutic product, but it may also be any other functional signal sequence, or an artificial signal sequence. The nucleic acid may also comprise a signal sequence which directs the synthesized therapeutic product towards a specific compartment of the cell.

The compositions according to the present invention may be prepared by mixing an aqueous solution which contains a mineral particle having an exchangeable lamellae structure with a solution which contains the nucleic acids. More precisely, the compositions according to the present invention may be prepared by dissolving the mineral particles having an exchangeable lamellae structure in an aqueous solution at a pH which is close to neutral (for example between 6 and 8, and more preferably between 6.5 and 7.5), then adding the aqueous solution thus obtained to a solution containing the nucleic acids or alternatively by adding the said solution containing the nucleic acids to the aqueous solution of mineral particles having an exchangeable lamellae structure. The solution containing the nucleic acids is preferably an isotonic solution in sodium chloride or in glucose. Preferably, the mineral particles having an exchangeable lamellae structure and the nucleic acids are introduced into the composition in quantities such that the mass ratio between the mineral particles having an exchangeable lamellae structure and the nucleic acids is between 0.01 and 1 000, preferably between 0.01 and 500, more preferably between 0.01 and 100, and even more preferably between 0.5 and 100. In any case, the respective quantities of each component may be adjusted and easily optimized by persons skilled in the art by the conventional method of trial and error, according to the mineral particle having an exchangeable lamellae structure which is used, the nucleic acid and the applications sought (in particular the cell type to be transfected).

A subject of the invention is also the compositions as defined above for use on a medicament.

A subject of the invention is also the use of the compositions as defined above for the transfer of nucleic acids into the cells in vitro, in vivo or ex vivo. More precisely, a subject of the present invention is the use of the compositions as defined above for the preparation of a medicament which is intended to treat diseases, in particular diseases which result from a deficiency in a protein or nucleic product. The nucleic acid contained in the said medicament encodes the said protein or nucleic product, or constitutes the said nucleic product, which is capable of correcting the said diseases in vivo or ex vivo. The said medicament may, in addition, be a vaccine, and in this case, the transfected nucleic acids induces an immune response.

For in vivo uses, for example in therapy or for the study of gene regulation or the creation of animal models of pathologies, the compositions according to the invention may be formulated with a view to administration in particular via the topical, cutaneous, oral, rectal, vaginal, parenteral, intranasal, intravenous, intramuscular, subcutaneous, intraocular, transdermal, intratracheal or intraperitoneal route. Preferably, the compositions of the invention contain a vehicle which is pharmaceutically acceptable for an injectable formulation, in particular for a direct injection into the desired organ, or for administration via the topical route (on the skin and/or on the mucous membrane). They may in particular be isotonic sterile solutions, or dry, in particular lyophilized, compositions which, upon addition, depending on the case, of sterilized water or of physiological saline, allow the constitution of injectable solutions. In addition, the compositions according to the invention may contain an adjuvant or adjuvants which are conventionally used in pharmacy. The nucleic acid doses used for the injection as well as the number of administrations may be adapted according to various parameters, and in particular according to the mode of administration used, the pathology concerned, the gene to be expressed, or alternatively the desired duration of treatment. As regards more particularly the mode of administration, it may be either a direct injection into the tissues, for example at the level of the tumours, or into the circulatory system, or a treatment of cells in culture followed by their reimplantation in vivo, by injection or transplantation. The relevant tissues in the context of the present invention are for example the muscles, skin, brain, lungs, liver, spleen, bone marrow, thymus, heart, lymph, blood, bones, cartilages, pancreas, kidneys, bladder, stomach, intestines, testicles, ovaries, rectum, nervous system, eyes, glands or connective tissues.

Another subject of the present invention relates to a method for transferring nucleic acids into the cells comprising the following steps:

(1) the formation of a composition comprising at least one nucleic acid and one mineral particle having an exchangeable lamellae structure as defined above, and

(2) bringing the cells into contact with the composition formed in (1).

More particularly, the present invention relates to a method of therapeutic treatment of the human or animal body comprising the following steps: (1) the formation of a composition comprising at least one nucleic acid and one mineral particle having an exchangeable lamellae structure as defined above, and (2) bringing the cells of the human or animal body into contact with the composition formed in (1).

The cells may be brought into contact with the composition according to the present invention by incubating the cells with the said composition or by injecting the composition into the organism or a part of the organism. The incubation is carried out preferably in the presence, for example, of 0.01 to 1000 μg of nucleic acid per 10⁶ cells. For administration in vivo, nucleic acid doses ranging from 0.01 to 10 mg may for example be used.

The compositions of the invention may, in addition, contain one or more pharmaceutically acceptable adjuvants. In this case, the adjuvant(s) is (are) previously mixed with the aqueous solution containing the mineral particle having an exchangeable lamellae structure according to the invention and/or with the solution of nucleic acid(s).

The present invention thus provides a particularly advantageous method for transferring nucleic acids, in particular for the treatment of diseases, comprising the administration in vivo or ex vivo of a nucleic acid which encodes a protein or can be transcribed into a nucleic acid capable of correcting the said disease, the said nucleic acid being combined with a mineral particle having an exchangeable lamellae structure as defined above under the conditions defined above.

The compositions according to the present invention are particularly useful for transferring nucleic acids into primary cells or into established lines. They may be for example fibroblast cells, muscle cells, nerve cells (neurons, astrocytes, glial cells), hepatic cells, haematopoietic cells (for example lymphocytes, CD34, or dendrite cells) or epithelial cells, in differentiated or pluripotent (precursors) form.

In addition to the preceding arrangements, the present invention also comprises other characteristics and advantages which will emerge from the examples and figures below, and which should be considered as illustrating the invention without limiting its scope.

### FIGURES

FIG. 1: idealized structure of the mineral particles having an exchangeable lamellae (lamellar double hydroxide) structure according to the present invention.

FIG. 2: schematic representation of hydrotalcite Mg₆Al₂CO₃(OH)₁₆.4H₂O.

FIG. 3: measurement of the zeta potential of hydrotalcite particles alone in solution.

FIG. 4: measurement of the zeta potential of compositions containing hydrotalcite particles and DNA in a mass ratio equal to 0.5.

FIG. 5: Measurement of the percentage of DNA remaining in the supernatant relative to the initial quantity of DNA introduced, as a function of the hydrotalcite/DNA mass ratio. The measurements were carried out for three different concentrations of DNA introduced: 20 μg of DNA/ml, 50 μg of DNA/ml, and 100 μg of DNA/ml.

FIG. 6: Agarose gel electrophoresis of different formulations of DNA. The column “a” constitutes the control (DNA which is unformulated and not subjected to nucleases), the column “b” represents the unformulated DNA which is subjected to nucleases, and the columns “c” to “f” represent hydrotalcite/DNA compositions at mass ratios of 0.125 or 0.25 or 0.5 or 1 which are subjected to nucleases.

FIG. 7: Visualization in the form of a histogram of the efficiency of gene transfer in vivo (by injection into the cranial tibial muscle of mice) of DNA/hydrotalcite compositions in different mass ratios, compared to the injection of naked DNA.

FIG. 8: Schematic representation of the pXL3031 plasmid used in the experiments of DNA transfer into the cells.

### EXAMPLES

A] Materials and Methods

Mice Used:

The mice used in the experiments of in vivo gene transfer are female C57B16 mice which are 8 weeks old, and divided up into 9 groups of 12 mice.

Mineral Particle According to the Invention Used

The mineral particle having an exchangeable lamellae structure used in the different tests is hydrotalcite in aqueous solution at a concentration of 20 g/l and at pH 7. This hydrotalcite is sold by the company Süd-Chemie AG (Germany).

DNA

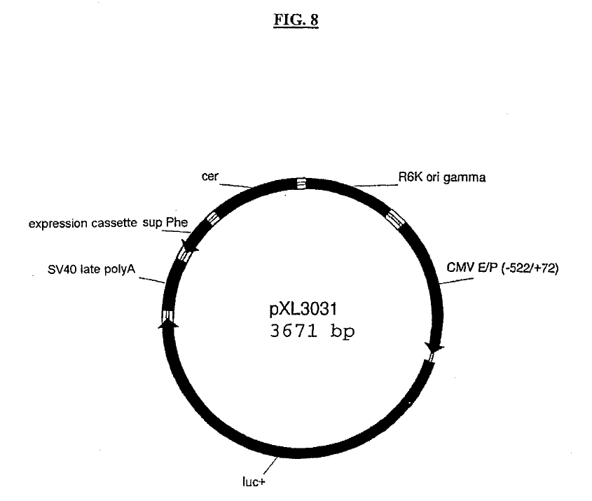

The plasmid used is pXL3031 which contains the luc gene encoding luciferase under the control of the P/E CMV promoter of the cytomegalovirus, represented in FIG. 8. Its size is 3671 bp. The plasmid solution used contains 320 μg of DNA/ml in a solution of sodium chloride (NaCl) at 150 mM.

Hydrotalcite/DNA Complexes

The complexes are prepared by mixing equal volumes of two solutions: one containing the hydrotalcite and the other the plasmid DNA.

### Example 1

Measurement of the Zeta Potentials of the Hydrotalcite Alone and of the DNA/Hydrotalcite Complexes

The aim of this example is to show that the DNA is capable of associating with a mineral particle having an exchangeable lamellae structure, such as hydrotalcite. The measurement of the zeta potential provides information on the nature of the surface charge of a particle placed in a liquid. The zeta potential is not a direct measurement of the surface charge, but is the potential which exists around the particle when this particle is displaced in a solution when it is subjected to an electric field. Consequently, if the particles have a positive surface charge, the zeta potential, which is the only measurable parameter, will itself also be positive. The zeta potential plays a determining role in the colloidal stability of particles in solution. Specifically, when the zeta potential is highly positive or negative, the particles do not aggregate because electrostatic forces of repulsion keep them isolated. On the other hand, particles which possess a zeta potential close to zero are not stable because the Van Der Waals forces mutually attract the particles, and make them precipitate.

The zeta potential of the hydrotalcite and of the hydrotalcite/DNA complexes was measured using a Coulter zetameter (Delsa 440 SX). The hydrotalcite solution contained 0.15 mg of hydrotalcite/ml in a 20 mM solution of sodium chloride (NaCl), i.e. a conductivity of 2.3 mS/cm. The hydrotalcite/DNA complexes were prepared at concentrations of 250 μg of DNA/ml in a 20 mM solution of sodium chloride (NaCl), at the mass ratio of 0.5. The measurement was carried out by applying an electrical field of 1.5 mA for 60 seconds.

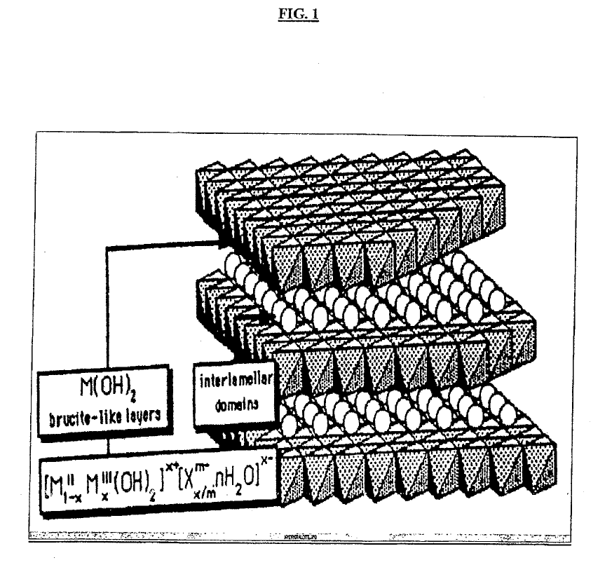

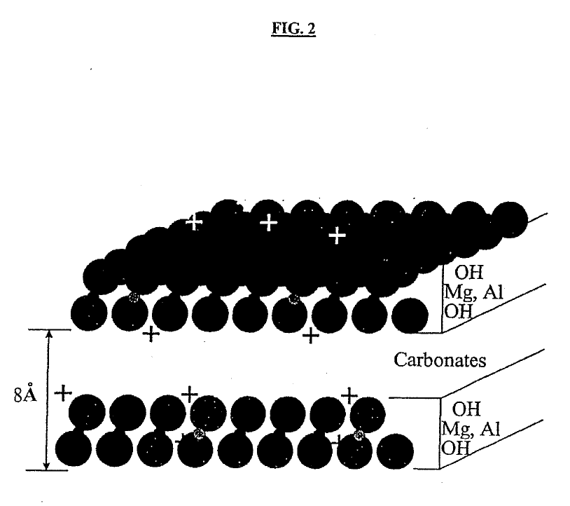

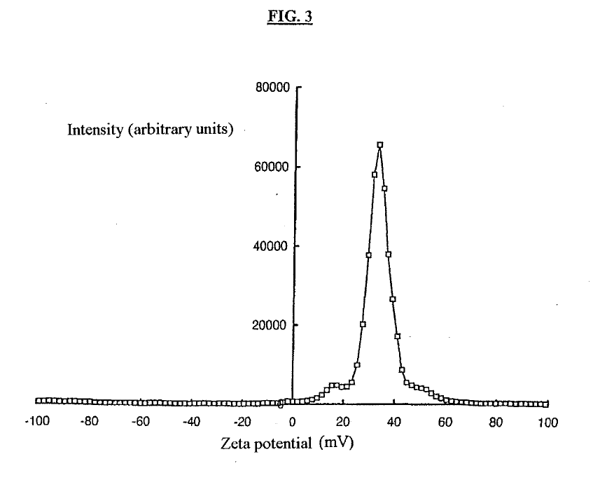

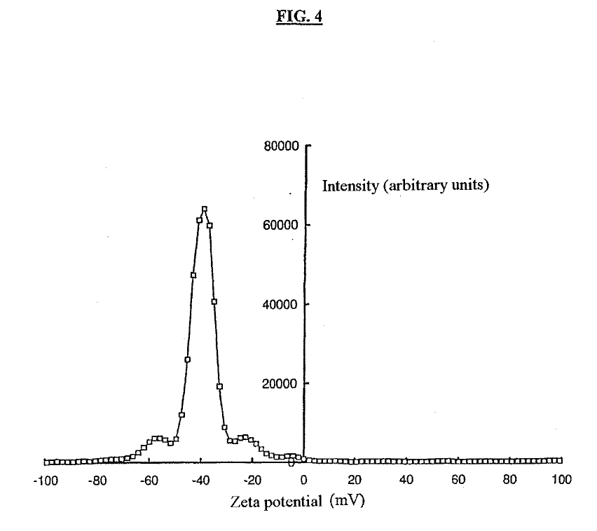

FIG. 3 represents the zeta potential of the hydrotalcite alone before being mixed with the DNA. This zeta potential is +32 mV and thus indicates an overall cationic surface charge, which is coherent with the structure and the composition of hydrotalcite (see FIGS. 1 and 2). FIG. 4 represents the zeta potential of hydrotalcite/DNA complexes at the mass ratio of 0.5. In this case, the zeta potential is −41 mV. The surface charge has, therefore, been modified and is now globally anionic, which indicates that the anionic DNA molecules have joined together on the surface of the hydrotalcite, thus modifying its surface charge.

### Example 2

Demonstration of the Association Between DNA and the Mineral Particles Having an Exchangeable Lamellae Structure Such as Hydrotalcite

The aim of this example is to show that the mineral particles having an exchangeable lamellae structure such as hydrotalcite associate with the DNA.

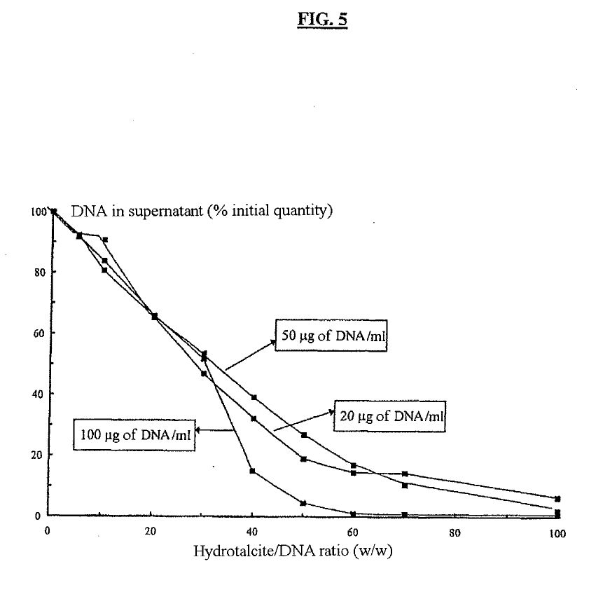

Adsorption isotherms are produced by mixing increasing quantities of hydrotalcite with a constant quantity of DNA. The hydrotalcite/DNA mixes are prepared by mixing equal volumes of a solution containing the DNA and a solution containing the hydrotalcite. All these mixes together are then ultracentrifuged at 50,000 revs/minute for 10 minutes (ultracentrifuge: Beckman TL100). The graph in FIG. 5 indicates that the amount of DNA present in the supernatant progressively diminishes as the hydrotalcite/DNA mass ratio increases. At a hydrotalcite/DNA ratio of 50 weight/weight, there is no more DNA present in the supernatant. This indicates that all the DNA molecules are associated with the hydrotalcite and are to be found in the pellet after ultracentrifugation. The same phenomenon has been observed at three different concentrations of DNA: 20, 50 and 100 μg of DNA/ml. Consequently it may be deduced that there is indeed association between the hydrotalcite and the DNA.

### Example 3

Protection of the DNA with Regard to Degradation Caused by Nucleases

The aim of this example is to show that the DNA is protected with regard to enzymatic degradation when it is associated with a mineral particle having an exchangeable lamellae structure such as hydrotalcite.

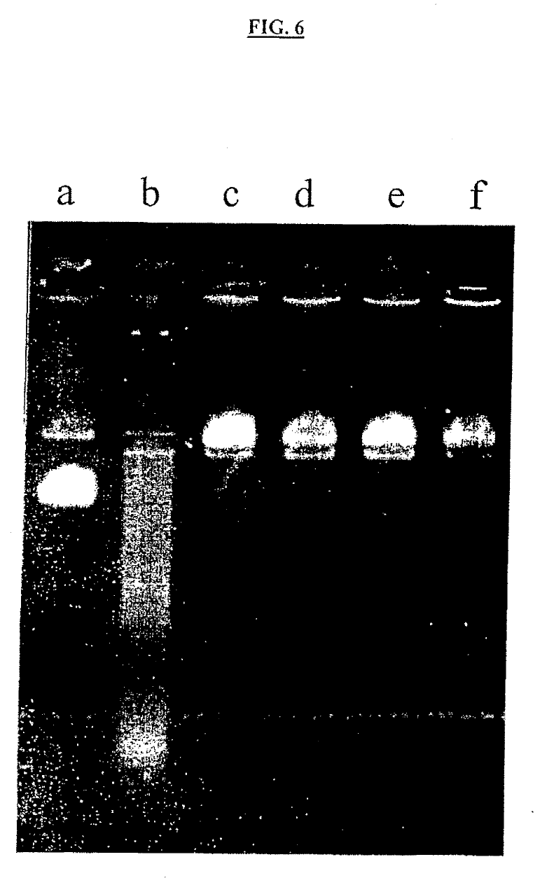

Samples of naked DNA and of DNA formulated with hydrotalcite were subjected to the test of resistance to degradation caused by nucleases. For this, the samples were incubated with a sample of muscle fluid. FIG. 6 indicates that when the DNA is associated with the hydrotalcite at different mass ratios (columns “c”, “d”, “e” and “f”), it is not degraded by the nucleases since it is entirely possible to observe a DNA band on the gel. On the other hand the column “b” which represents the naked DNA which is not associated with the hydrotalcite is totally degraded by the nucleases because instead of having a well identified DNA band on the gel, a multitude of small fragments which migrate in the gel are observed.

Thus, when the DNA is associated with the hydrotalcite mineral particles, it is protected from the degradation action of the nucleases, as opposed to the naked DNA which is rapidly degraded. This property which is conferred by the mineral particles having an exchangeable lamellae structure such as hydrotalcite is very advantageous because a greater amount of DNA can thus reach the nucleus of the cells so as to be transcribed there, with the beneficial consequences that this involves in terms of efficiency of transfection.

### Example 4

Transfection in vivo of Hydrotalcite/DNA Complexes

a) Injections

The injections are carried out bilaterally into the cranial tibial muscle of the mice in a proportion of 25 μl/muscle, i.e. 4 μg of DNA/muscle.

The animals are anaesthetized with 250 μl of Ketamine/Xylazine by intraperitoneal route (3.9 ml of imalgene 1000, 0.6 ml of 2% Rompun, and 40.5 ml of a 150 mM solution of sodium chloride NaCl). Next, the animals are shaved and injected at the level of the two cranial tibial muscles.

b) Removal of the Muscles

The cranial tibial muscles are recovered 6 days post-injection in 1 ml of lysis buffer/muscle (Lysis Buffer from Promega E) supplemented with protease inhibitors (coktail tablets—Boehringer). The muscles are removed into special tubes from BIO101 and conserved at −20° C., before crushing and reading.

c) Extraction of the Luciferase Expressed by the Muscle Cells

Extraction of the luciferase is carried out with a Fastprep machine under extraction conditions using 1 ml of lysis buffer per tube at a speed of 6.5 m/s, for 30 seconds. The tubes are then put on ice and centrifuged at 12,000 g for 10 minutes at 4° C.

d) Determination of Luciferase Activity

The luciferase activity is measured using a luciferase test kit (Promega) and a Dynex MLX luminometer. The reading for this activity is measured in RLU (Relative Light Unit).

e) Transfection in vivo in the Muscle

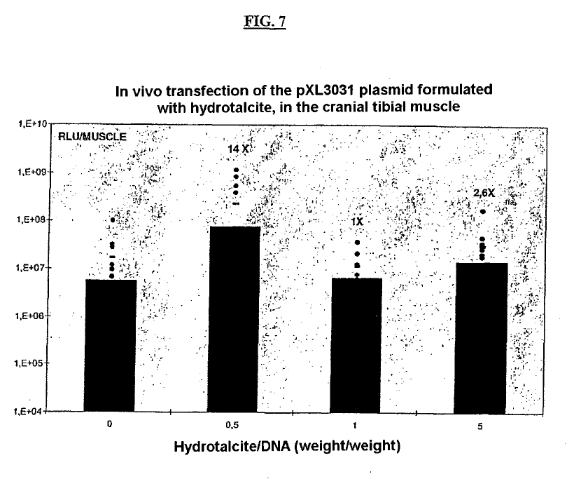

The results of gene transfer in the muscle in vivo are represented in FIG. 7. These results indicate that at the hydrotalcite/DNA mass ratio of 0.5 an increase in the luciferase activity of a factor of 14 is obtained with respect to naked DNA. More generally, the hydrotalcite/DNA compositions have an improved efficiency of transfection with respect to that obtained by injection of naked DNA.

1. Composition comprising at least one nucleic acid and one mineral particle having an exchangeable lamellae structure with the exclusion of the compositions comprising a deoxyribonucleic acid having a size of less than 1000 base pairs.

2. Composition comprising at least one nucleic acid having a size greater than 1 000 base pairs and a mineral particle having an exchangeable lamellae structure.

3. Composition comprising at least one ribonucleic acid and a mineral particle having an exchangeable lamellae structure.

4. Composition according to claim 1 or 2, characterized in that the nucleic acid is a plasmid, a vector, an episome or an expression cassette.

5. Composition according to one of claims 1 to 4, characterized in that the said mineral particle having an exchangeable lamellae structure has as a general formula:
[M₁₋ₓ^{II}Mₓ^{III}(OH)₂]^{x+}[X_{x/m}^{m-}.nH₂O]^{x-}
in which M^{II} represents a divalent metal cation, M^{III}represents a trivalent metal cation, X^{m-} represents an interfoliar anion and m is an integer greater than or equal to 1, x is strictly between 0 and 1, and n is strictly greater than 0.

6. Composition according to claim 5, characterized in that the M^{II} divalent metal cation may be chosen from magnesium (Mg), chromium (Cr), manganese (Mn), iron (Fe), nickel (Ni), cobalt (Co), copper (Cu) or zinc (Zn).

7. Composition according to claim 5, characterized in that the M^{II} divalent metal cation is magnesium.

8. Composition according to claim 5, characterized in that the M^{III} trivalent metal cation may be chosen from aluminium (Al), chromium (Cr), manganese (Mn), iron (Fe), nickel (Ni), cobalt (Co) or gallium (Ga).

9. Composition according to claim 5, characterized in that the M^{III} trivalent metal cation is aluminium.

10. Composition according to claim 5, characterized in that the X^{m-} interfoliar anion may be chosen from halides, oxoanions, iso- and heteroanions, complex anions, or organic anions.

11. Composition according to claim 5 or 10, characterized in that the X^{m-} interfoliar anion is the CO₃²⁻ carbonate.

12. Composition according to claim 5, characterized in that m is equal to 1, 2, 3, 4, 5 or 6.

13. Composition according to claim 5, characterized in that x is between 0.05 and 0.95.

14. Composition according to claim 5, characterized in that n is greater than or equal to 0.1.

15. Composition according to one of claims 1 to 5, characterized in that the said mineral particle having an exchangeable lamellae structure is chosen from hydrotalcite of formula Mg₆Al₂CO₃(OH)₁₆.4H₂O, manasseite of formula Mg₆Al₂CO₃(OH)₁₆.4H₂O, pyroaurite of formula Mg₆Fe₂CO₃(OH)₁₆.4H₂O, stichtite of formula Mg₆Cr₂CO₃(OH)₁₆.4H₂O, takovite of formula Ni₆Al₂CO₃(OH)₁₆.4H₂O, reevesite of formula Ni₆Fe₂CO₃(OH)₁₆.4H₂O or comblainite of formula Ni₆CO₂CO₃(OH)₁₆.4H₂O.

16. Composition according to claim 15, characterized in that the said mineral particle having an exchangeable lamellae structure is hydrotalcite of formula Mg₆Al₂CO₃ (OH)₁₆.4H₂O.

17. Composition comprising at least one nucleic acid and a mineral particle having an exchangeable lamellae structure of formula
[M₁₋ₓ^{II}Mₓ(OH)₂]^{x+}[X_{x/m}^{x-}.nH₂)]^{x-}
in which M^{II} represents magnesium, M^{III} represents aluminium, X^{m-} represents an interfoliar carbonate anion and m is an integer greater than or equal to 1, x is strictly between 0 and 1, and n is strictly greater than 0.

18. Composition according to claim 17, comprising at least one nucleic acid and one mineral particle having an exchangeable lamellae structure of formula Mg₆Al₂CO₃(OH)₁₆.4H₂O.

19. Composition according to one of claims 1 to 18, characterized in that it comprises, in addition, one or more pharmaceutically acceptable adjuvants.

20. Composition according to one of claims 1 to 19, characterized in that it comprises, in addition, a vehicle which is pharmaceutically acceptable for an injectable formulation.

21. Composition according to one of claims 1 to 19, characterized in that it comprises, in addition, a vehicle which is pharmaceutically acceptable for an application to the skin and/or the mucous membranes.

22. Composition according to one of claims 1 to 21, characterized in that the mineral particle having an exchangeable lamellae structure/nucleic acid(s) mass ratio is between 0.01 and 1000.

23. Composition according to one of claims 1, 2, 4, 17 or 18, characterized in that the nucleic acid has a size of between about 1 000 base pairs and about 2 000 000 base pairs.

24. Composition according to one of claims 1, 2, 4, 17 or 18, characterized in that the nucleic acid has a size of between about 1 000 base pairs and about 100 000 base pairs.

25. Composition according to one of claims 1, 2, 4, 17 or 18, characterized in that the nucleic acid has a size of between about 1 000 base pairs and about 20 000 base pairs.

26. Composition according to one of claims 1 to 25 for use as a medicament.

27. Use of a composition as defined in one of claims 1 to 26 for the preparation of a medicament which is intended for the transfection of cells.

28. Method for preparing a composition as defined in one of claims 1 to 26, characterized in that an aqueous solution which contains a mineral particle having an exchangeable lamellae structure is mixed with a solution which contains the nucleic acid(s).

29. Method of preparation according to claim 28, characterized in that, when the compositions as defined in the preceding claims contain, in addition, one or more pharmaceutically acceptable adjuvants, the adjuvant(s) is(are) previously mixed with the aqueous solution containing a mineral particle having an exchangeable lamellae structure and/or with the solution of nucleic acid(s).

30. Method for transferring nucleic acids into cells in vitro or ex vivo comprising the following steps:
(1) the formation of a composition comprising at least one nucleic acid and one mineral particle having an exchangeable lamellae structure as defined in one of claims 1 to 26, and
(2) bringing the cells into contact with the composition formed in (1).
